(19) United States
(12) Patent Application Publication
(10) Pub. No.: US 2006/0135918 A1
(43) Pub. Date: Jun. 22, 2006

(54) Reusable drug delivery device

(75) Inventor: Anthony LaRuffa, Rochester, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/301,559

(22) Filed: Dec. 13, 2005

Related U.S. Application Data
(60) Provisional application No. 60/638,481, filed on Dec. 22, 2004.

(51) Int. Cl. A61M 35/00

(52) U.S. Cl. 604/294

(57) ABSTRACT

This invention relates to a reusable drug delivery device, preferably a device that is placed or implanted in the eye to release a pharmaceutically active agent to the eye. The device utilizes a rigid box-like holder and cap and can be reused once the active has completely diffused from the core or upon completion of a research study.

### CROSS REFERENCE

This application claims the benefit of Provisional Patent Application No. 60/638,481 filed Dec. 22, 2004 and is incorporated herein by reference.

### FIELD OF THE INVENTION

This invention relates to a reusable drug delivery device, preferably a device that is placed or implanted in the eye to release a pharmaceutically active agent to the eye. The device utilizes a rigid box-like holder and cap and can be reused once the active has completely diffused from the core or upon completion of a research study.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye for treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core having a pharmaceutically active agent, and some type of holder for the drug core made of an impermeable material such as silicone or other hydrophobic materials. The holder includes one or more openings for passage of the pharmaceutically active agent through the impermeable material to eye tissue. Many of these devices include at least one layer of material permeable to the active agent, such as polyvinyl alcohol (PVA).

The prior art devices have been designed for single use and may require a complicated assembly process. This can provide difficulty in controlling certain studies that require relatively exact reproducibility. Moreover, it may be desirable to test a drug core in a laboratory environment with a device that does not require a complicated assembly process. Additionally, there remains a need for a device that is micromachined to fine tolerances that provides for improved accuracy of clinical research. Therefore there remains a need for reusable drug delivery devices for implantation in the eye.

### BRIEF DESCRIPTION OF THE DRAWINGS

FIG. 1 is a top plan view of a first embodiment of a drug delivery device of this invention.

FIG. 2 is a side plan view of the device of FIG. 1.

FIG. 3 is a perspective view of the device of FIG. 1.

FIG. 4 is a top plan view of the cap of a drug delivery device.

FIG. 5 is a side cross-section view of the cap of FIG. 4.

FIG. 6 is a perspective view of the cap of FIG. 4.

FIG. 7 is an exploded view of one embodiment of the device of this invention.

FIG. 8 is a perspective view of the assembled device of this invention.

FIG. 9 is a side cross-section view of the device showing the drug core.

### SUMMARY OF THE INVENTION

According to a first embodiment, this invention relates to a reusable drug delivery device. The reusable drug delivery device comprises a holder and a cap; the holder being capable of holding a drug core and as an anchoring mechanism for implantation, the holder being made of a material capable of being micromachined, stamped or formed and having an outside surface configured to reversibly receive a cap. A second embodiment of the invention comprises an anchoring mechanism for implantation thereby providing a unitary drug holder and anchoring mechanism for implantation and being made of a material capable of being micromachined, stamped, or formed. The anchoring mechanism for implantation (e.g., suture tab) contains a suture hole at one end of the device or other means for holding the device in place once implanted-.

### DETAILED DESCRIPTION OF PREFERRED EMBODIMENTS

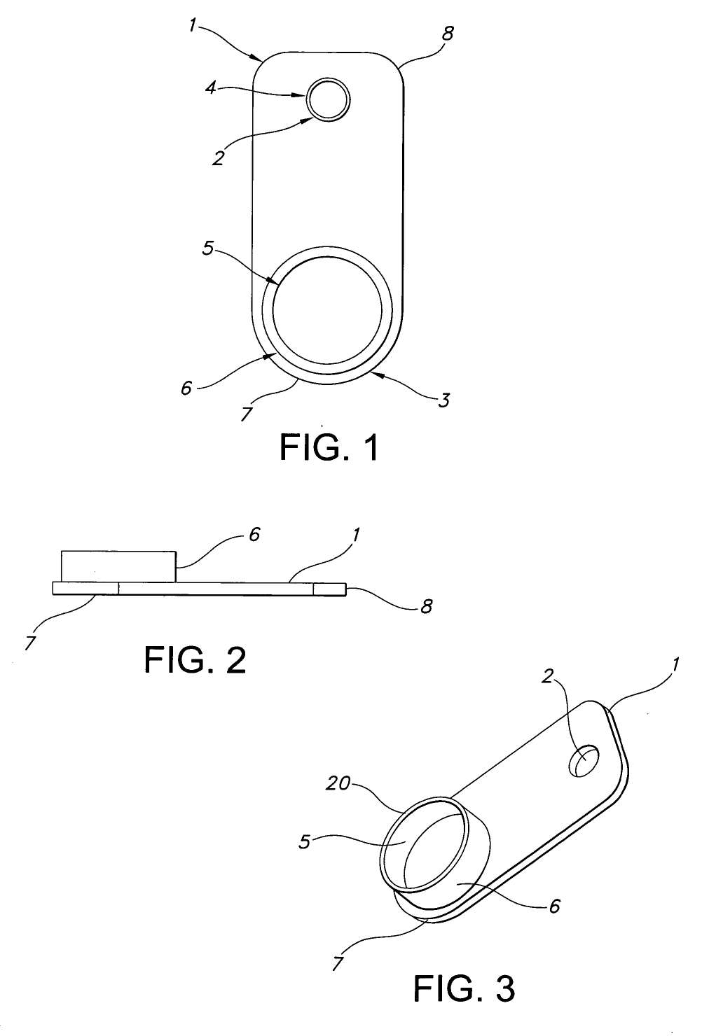

FIG. 1 is a top plan view of an embodiment of the base 1 of the device 50. The base 1 is generally elongate with a suture hole 2 at an end opposite the box-like holder 3 for the drug core (not shown). Suture hole 2 preferably comprises chamfered edges to avoid tearing of any suture material that may be used with the device. Holder 3 has an inside dimension 5 that is wide enough to hold the drug core and any optional coating(s). Holder 3 also has an outer side surface 6 that allows it to reversibly receive a cap 11. The structural relationship between outer side surface 6 and cap 11 will be explained further on in the description.

FIG. 2 is a side plan view of the device of FIG. 1. As shown in FIG. 2, the ends of base 1 can be curved 7, 8 to avoid sharp edges that may undesirably interact with tissue upon implantation. FIG. 3 is a perspective view of the device of FIG. 1.

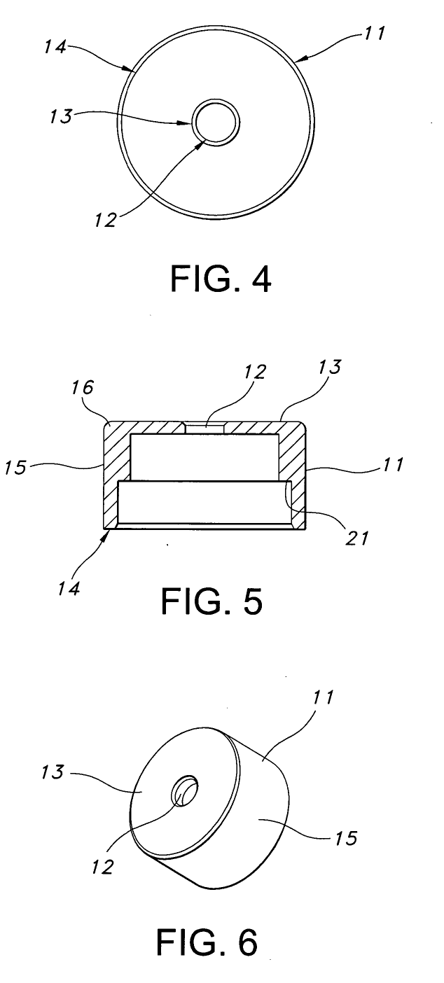

FIG. 4 is a top plan view of the cap 11 to be used with the base 1 of FIGS. 1-3. The cap 11 has an opening 12 to allow to release of the active found in drug core (not shown). Cap 11 also comprises a surface portion 13 that is impermeable to the agent. As indicated above, the cap 11 has an inner side surface 14 that reversibly engages with the outer side surface 6 of the holder 3 of the base 1. This inner side surface 14 is more easily seen in FIG. 5 which is a side cross-sectional view of cap 11. Cap 11 has an outer side surface 15 that is chamfered 16 at the shoulder where it joins the top surface 13 of the cap. As shown in FIG. 5, various internal shapes may be formed to accommodate the drug core (not shown) so long as inner side surface 14 is configured to removably engage with outer side surface 6 of the holder 3. By reversibly engage we mean that the cap 11 and holder 3 are able to form a seal that can be released when desired. The seal is accomplished through mechanical means such as a friction fit.

FIG. 6 is a perspective view of cap 11 showing opening 12 in the top surface portion 13 of cap 11. Also shown is the exterior side wall 15 of cap 11.

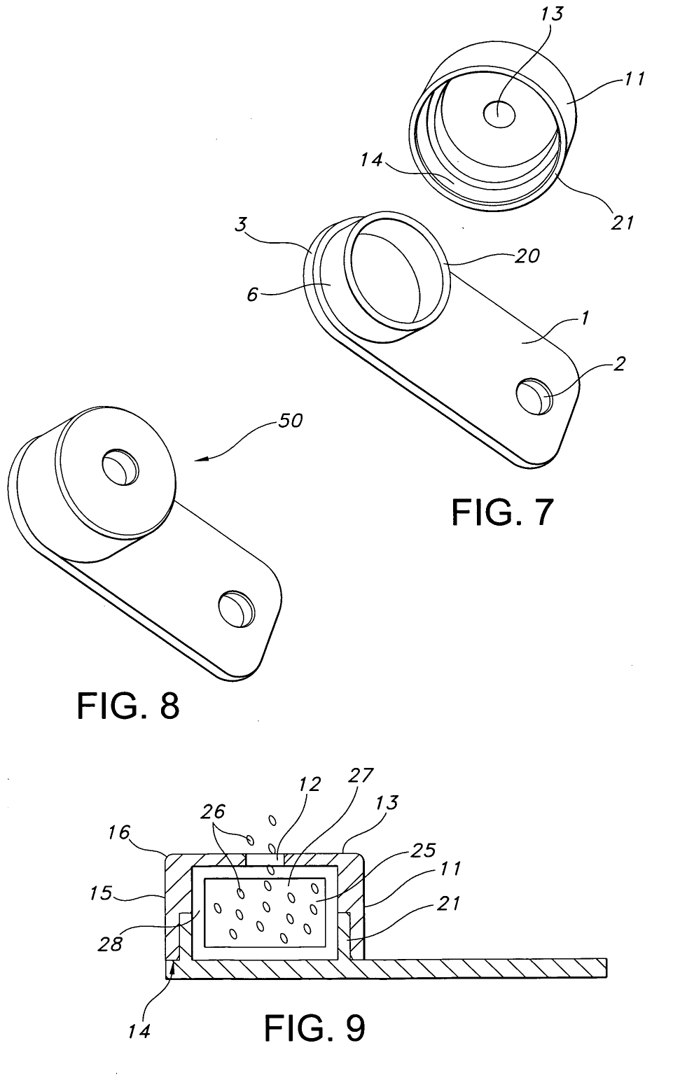

FIG. 7 is an exploded perspective view of the complete device 50 better showing how the top surface 20 of holder 3 is sized and configured to tightly engage with the inside bottom surface 21 of cap 11 to form a seal. In this embodiment, sealing is obtained not just through engagement of outer side surface 6 of the holder 3 with inner side surface 14 of cap 11 but also through engagement of top surface 20 of holder 3 with inside bottom surface 21 of cap 11.

FIG. 8 is a perspective view of the assembled device 50.

FIG. 9 is a side cross-sectional view of device 50 showing core 25 comprising a pharmaceutically active agent 26 contained in a matrix 27. The core 25 may optionally comprise a coating 28 which can assist in providing desirable release kinetics.

Although the embodiment shown utilizes a friction fit to allow the cap 11 to be removably attached to the holder 3 other engagements means are also envisioned. For example, outer side surface 15 and inner side surface 14 may be threaded to allow cap 11 to be screwed onto holder 3. Other engagement means would include threaded, pressed, locking or, in the absence of engagement means, sealed with an impermeable material. Also, although the preferred embodiment contains an elongated base with an opening to serve as a suture tab, this feature is not necessary for the successful operation of the device.

The active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecamil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility agents impeding such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal antiinflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and antineovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; antiinflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eseridine salicylate, carbachol, diisopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

Active agent 26 may be mixed with a matrix material 27. Preferably, matrix material is a polymeric material that is compatible with body fluids and the eye. Additionally, matrix material should be permeable to passage of the active agent therethrough, particularly when the device 50 is exposed to body fluids. For this embodiment, the matrix material is PVA. Also, in this embodiment, inner drug core may be coated 28 with a coating of additional matrix material which may be the same or different from material mixed with the active agent. For the illustrated embodiment, the coating employed is also PVA.

In addition to the illustrated materials, a wide variety of materials may be used to construct the devices of the present invention. The only requirements are that they are inert, non-immunogenic, of the desired permeability, and capable of being micro-machined. Materials that may be suitable for fabricating the device include naturally occurring or synthetic materials that are biologically compatible with body fluids and body tissues, and essentially insoluble in the body fluids with which the material will come in contact and capable of being micro-machined. The use of rapidly dissolving materials, materials highly soluble in body fluids, or highly flexible materials are to be avoided since dissolution of the wall would affect the constancy of the drug release, as well as the capability of the device 50 to remain in place for a prolonged period of time and flexible materials may be difficult to machine.

Naturally occurring or synthetic materials that are biologically compatible with body fluids and eye tissues and essentially insoluble in body fluids which the material will come in contact include, but are not limited to metal, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

A device of the type shown in FIGS. 1-9 may be manufactured as follows. Standard micromachining techniques such as milling, lathing, etching, etc. are applied to the material used to form device 50. Such techniques are within the purview of one of ordinary skill in the art.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

It should be understood that the preferred device comprises a suture tab. However, a suture tab is not necessary for therapeutic operation of the device.

The device is typically provided to the end user in a sealed sterilized package, for example, by gamma irradiation, for example, such as is disclosed in U.S. patent application Ser. No. 10/183,804, the contents of which are incorporated by reference herein.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A reusable drug delivery device, comprising:
a holder and a cap;
the holder comprising a drug core, the holder being made of a material capable of being micromachined and having an outside surface configured to reversibly receive a cap.

2. The device of claim 1, wherein the cap comprises a surface portion that is impermeable to the active, the surface having an opening to allow the release of the active found in drug core.

3. The device of claim 1, wherein the cap has an inner surface that reversibly engages with an outer surface of the holder.

4. The device of claim 1, further comprising a suture tab anchoring device integral with the holder.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder has an inside circumference that is wide enough to hold the drug core and any coating if present.

8. The device of claim 1, wherein the drug core is cylindrical.

9. The device of claim 1, wherein the drug core is coated with a material permeable to said active agent.

10. The device of claim 1, comprising a mixture of pharmaceutically active agents.

11. The device of claim 1 sized and configured so as to be suitable for implantation in the human eye.
